(19) United States
(12) Patent Application Publication
(10) Pub. No.: US 2024/0398299 A1
(43) Pub. Date: Dec. 5, 2024

(54) METHODS AND SYSTEMS FOR POINT OF CARE EXOSOME ISOLATION

(71) Applicant: CROWN LABORATORIES, INC., Johnson City, TN (US)

(72) Inventors: Thomas M. HITCHCOCK, Dallas, TX (US); Brian Christopher JONES, Northlake, TX (US); Harish RAMACHANDRAMOORTHY, Arlington, TX (US)

(21) Appl. No.: 18/677,281

(22) Filed: May 29, 2024

Related U.S. Application Data
(60) Provisional application No. 63/504,956, filed on May 30, 2023.

(51) Int. Cl. A61B 5/15

(52) U.S. Cl. CPC A61B 5/150755

(57) ABSTRACT

Described herein are methods and systems for isolating exosomes or extracellular vesicles from a blood sample. In one embodiment, the method comprises isolating and fractionating whole blood and using beads to shear platelets and leukocytes causing the release of exosomes or extracellular vesicles. The resulting exosome-rich platelet rich plasma (ER-PRP) can be further refined by microfiltration.

### CROSS-REFERENCE TO RELATED APPLICATIONS

This application claims priority to U.S. Provisional Patent Application No. 63/504,956, filed on May 30, 2023, which is incorporated by reference herein in its entirety.

### BACKGROUND

Exosomes are found in blood and are released from a number of potential cell sources (e.g., mesenchymal stem cells, platelets). When blood is fractionated based on density, the plasma and platelet rich plasma (PRP) fractions contain exosomes and other extracellular vesicles (EVs). The quantity of EVs and exosomes can be significantly increased by activating platelets, such as with CaCl₂). However, platelet activation also initiates the clotting cascade which entraps the EVs and exosomes in a clot, limiting the ability to separate the exosomes out from the other components of the PRP.

What is needed are rapid methods and systems for isolating exosomes and extracellular vesicles from whole blood that can be used in a point of care environment for therapeutic and diagnostic applications.

### SUMMARY

One embodiment described herein is a method for isolating exosomes or extracellular vesicles from a blood sample, the method comprising: adding a blood sample to an isolation container comprising: a physical barrier configured to separate non-red blood cell blood components from red blood cells; a plurality of particles having a particle density that is less than a density of the physical barrier, the plurality of particles being configured to generate shear stress on non-red blood cell blood components; and an anticoagulant; centrifuging the isolation container to separate non-red blood cell blood components from red blood cells, thereby generating a platelet-rich plasma (PRP); and agitating the isolation container to generate shear stress on one or more non-red blood cell blood components and release exosomes or extracellular vesicles from the PRP, thereby generating an exosome-rich PRP (ER-PRP). In one aspect, the method, further comprises filtering the ER-PRP to remove platelets and generate an exosome-rich plasma (ERP). In another aspect, filtering comprises the use of a filter having pores ranging from about 0.1 μm to about 5 μm in size. In another aspect, the method, further comprises filtering the ER-PRP to remove white blood cells. In another aspect, filtering comprises the use of a filter having pores ranging from about 0.1 μm to about 20 μm in size. In another aspect, the isolation container comprises a glass tube or a plastic tube. In another aspect, the physical barrier comprises a valve, a polymer disc or buoy, or a thixotropic gel. In another aspect, the physical barrier comprises a density ranging from about 1.05 g/cm³ to about 1.10 g/cm³. In another aspect, the plurality of particles comprises a plurality of beads comprised of polypropylene, zirconium, or a combination thereof. In another aspect, the plurality of particles comprises a density ranging from about 0.95 g/cm³ to about 1.05 g/cm³. In another aspect, the anticoagulant comprises anticoagulant citrate dextrose solution A (ACDA), calcium citrate, or a combination thereof. In another aspect, centrifuging the isolation container comprises centrifugation at about 100-7000×g for about 5-60 minutes to separate non-red blood cell blood components from red blood cells. In another aspect, centrifuging the isolation container comprises centrifugation at about 1000-1500×g for about 10-20 minutes to separate non-red blood cell blood components from red blood cells. In another aspect, agitating the isolation container comprises agitation at an angle of about 30° to about 60° to generate shear stress on the one or more non-red blood cell blood components. In another aspect, agitating the isolation container comprises agitation at an angle of about 45° to generate shear stress on the one or more non-red blood cell blood components. Another embodiment described herein is exosomes or extracellular vesicles isolated using the methods described herein.

Another embodiment described herein is a method for isolating exosomes or extracellular vesicles from a platelet-rich plasma (PRP) sample, the method comprising: adding a PRP sample to an isolation container comprising: a plurality of particles configured to generate shear stress on non-red blood cell blood components; and an anticoagulant; and agitating the isolation container to generate shear stress on one or more non-red blood cell blood components and release exosomes or extracellular vesicles from the PRP sample, thereby generating an exosome-rich PRP (ER-PRP). In one aspect, the method, further comprises filtering the ER-PRP to remove platelets and generate an exosome-rich plasma (ERP). In another aspect, filtering comprises the use of a filter having pores ranging from about 0.1 μm to about 5 μm in size. In another aspect, the method, further comprises filtering the ER-PRP to remove white blood cells. In another aspect, filtering comprises the use of a filter having pores ranging from about 0.1 μm to about 20 μm in size.

Another embodiment described herein is exosomes or extracellular vesicles isolated using the methods described herein.

Another embodiment described herein is a system for isolating exosomes or extracellular vesicles from a blood sample, the system comprising: an isolation container comprising a physical barrier configured to separate non-red blood cell blood components from red blood cells in a blood sample; a plurality of particles having a particle density that is less than a density of the physical barrier, the plurality of particles being configured to generate shear stress on non-red blood cell blood components; and an anticoagulant. In one aspect, the system further comprises a centrifuge to separate non-red blood cell blood components from red blood cells. In another aspect, the system further comprises an agitator to generate shear stress on non-red blood cell blood components.

Another embodiment described herein is exosomes or extracellular vesicles isolated using the systems described herein.

Another embodiment described herein is the use of the systems described herein for the isolation of exosomes or extracellular vesicles.

Another embodiment described herein is a kit for isolating exosomes or extracellular vesicles from a blood sample, the kit comprising: an isolation container comprising: a physical barrier configured to separate non-red blood cell blood components from red blood cells in a blood sample; a plurality of particles having a particle density that is less than a density of the physical barrier, the plurality of particles being configured to generate shear stress on non-red blood cell blood components; and an anticoagulant; optionally, one or more of a filter, a hypodermic needle, or a syringe; optionally, buffers and receptacles; and optionally, one or more of packaging or instructions for use.

### DESCRIPTION OF THE DRAWINGS

FIG. 1 shows a conventional device and methodology for separating blood components to generate a Platelet-Rich Plasma (PRP) fraction.

FIG. 2A-B show schematics illustrating the generation of Exosome-Rich Platelet-Rich Plasma (ER-PRP) and Exosome-Rich Plasma (ERP) using exemplary methods and systems as described herein. FIG. 2A shows the use of an isolation container comprising a physical barrier and particles, and agitation to generate an ER-PRP from a blood sample. FIG. 2B shows that the ER-PRP can be further filtered to generate a more refined ERP fraction.

FIG. 3 shows exosome size measured by Nanoparticle Tracking Analysis. Data shown in Table 2.

FIG. 4 shows the exosome concentration measured by Nanoparticle Tracking Analysis. Data shown in Table 2.

FIG. 5 shows gene ontology functional analysis of differently expressed gene enrichment in exosomes isolated with PRP Beads compared to PRP Shake.

FIG. 6 shows proteomic analysis of differently expressed proteins in exosomes isolated with PRP Beads compared to PRP Shake.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, the term “sample” refers to a specimen or culture obtained from a subject. Biological samples can be obtained from subjects and encompass fluids, solids, tissues, and gases. In one embodiment, the subject's sample is a blood sample. Blood samples include whole blood, plasma, serum, blood products such as platelet-rich plasma, or fractionated blood components, such as one of the Cohn fractions I-IV, or an antibody fraction.

As used herein, “instructions for use” refers to a publication, diagram, or any other medium of expression which is used to provide instructions, information, or steps for performing the methods described herein. The instructions for use can be provided in printed form, affixed to a container which contains the kit materials, shipped together with the kit, or provided at an internet site. The information and instructions of the disclosed kits may be in the form of words, pictures, or both, and the like.

As used herein, the term “purified” means that a compound or entity is separated from other compounds or entities. A compound or entity (e.g., protein, peptide, cell, exosome) may be partially purified, substantially purified, or pure. A compound or entity is considered pure when it is removed from substantially all other compounds or entities (i.e., preferably at least about 90%). The term “isolated” and the phrase “biologically pure” refer to material which is substantially or essentially free from components which normally accompany it as found in its native state.

As used herein, the term “extracellular vesicles” (EVs) includes exosomes, microvesicles, and apoptotic bodies. Depending on their cellular site of origin, these vesicles have distinct structural and biochemical properties that affect their function and role in biological systems. For example, exosomes are generally homogeneous and are about 40-150 nm in size, while microvesicles and apoptotic bodies are heterogeneous in appearance and from 100-1000 nm and greater than 1000 nm in size, respectively.

As used herein, the term “exosome” refers to cell-derived vesicles having a diameter of between about 20-150 nm, such as between 40 and 120 nm, preferably a diameter of about 50-120 nm, for example, a diameter of about 70 nm, 80 nm, 90 nm, 100 nm, or 120 nm. Exosomes are also known in the art as “small extracellular vesicles” or “sEVs.” Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g. immature dendritic cells (wild-type or immortalized), induced and non-induced pluripotent stem cells, fibroblasts, platelets, immune cells, reticulocytes, tumour cells, mesenchymal stem cells, satellite cells, hematopoietic stem cells, pancreatic stem cells, white and beige pre-adipocytes and the like). As one of skill in the art will appreciate, cultured cell samples will be in the cell-appropriate culture media (using exosome-free serum). Exosomes include specific surface markers not present in other vesicles, including surface markers such as tetraspanins, e.g., CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein). Exosomes may also be obtained from a non-mammal or from cultured non-mammalian cells. As the molecular machinery involved in exosome biogenesis is believed to be evolutionarily conserved, exosomes from non-mammalian sources include surface markers which are isoforms of mammalian surface markers, such as isoforms of CD9 and CD63, which distinguish them from other cellular vesicles. As used herein, the tem “mammal” is meant to encompass, without limitation, humans, domestic animals such as dogs, cats, horses, cattle, swine, sheep, goats, and the like, as well as non-domesticated animals such as, but not limited to, mice, rats, and rabbits. The term “non-mammal” is meant to encompass, for example, exosomes from microorganisms such as bacteria, flies, worms, plants, fruit/vegetables, and yeast.

As used herein, the term “non-red blood cell blood components” refers to the components of whole blood that remain after red blood cells have been separated out, isolated, or removed. These non-red blood cell blood components include plasma, white blood cells (leukocytes), platelets, and combinations thereof which comprise less than whole blood. Such combinations may include, e.g., buffy coat depleted reconstituted blood, plasma-buffy coat, platelet-rich plasma (PRP), and the like.

As used herein, the term “platelets,” also known as “thrombocytes,” refer to the anucleate fragments of megakaryocytes involved in blood coagulation, hemostasis, and blood thrombus formation. Human platelets are routinely isolated through a variety of methods including platelet apheresis, plateletpheresis, gel filtration, or differential centrifugation. Platelets are about 2-3 μm in diameter and are filled with granules of growth factors and cytokines that are released when the platelet is activated. Adults have an average of about 200,000 platelets/μL blood sample.

As used herein, the term “plasma,” refers to the non-cellular component of uncoagulated blood. Plasma is comprised mainly of water, but also contains many important substances such as proteins (e.g., albumin, clotting factors, antibodies, enzymes, and hormones), sugars (e.g., glucose), and fat particles.

As used herein, the term “platelet-rich plasma” (PRP), refers to plasma which has a concentration of platelets at least as great as whole blood. “Platelet-poor plasma” (PPP) refers to plasma from which platelets have been removed in part or completely, where the PPP has a concentration of platelets less than whole blood. In some embodiments described herein, a PRP is generated by separating non-red blood cell blood components from red blood cells using centrifugation. In some embodiments described herein, a PRP comprises at least about 2×10⁵, 3× 10⁵, 4×10⁵, 5×10⁵, 6×10⁵, 7×10⁵, 8×10⁵, 9×10⁵, 1×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, or 2×10⁶ platelets/μL.

As used herein, the term “exosome-rich PRP” (ER-PRP), refers to plasma which has a concentration of released exosomes or extracellular vesicles higher than that of the PRP from which it is derived. In some embodiments described herein, an ER-PRP is generated by applying shear stress to one or more non-red blood cell blood components of a PRP sample. In some embodiments described herein, an ER-PRP may further be filtered to remove white blood cells and/or platelets.

As used herein, the term “exosome-rich plasma” (ERP), refers to an ER-PRP sample that has platelets removed in part or completely. In some embodiments described herein, an ERP is generated by filtering an ER-PRP sample. In some embodiments described herein, an ERP may further be filtered to remove white blood cells.

As used herein, the term “isolation container” refers to any container suitable for isolating exosomes or EVs from a blood sample or a PRP sample. In some embodiments, the isolation container may comprise a suitable type of glass tube or plastic tube, or any other suitable isolation container well-known in the art and well-equipped for handling blood and PRP samples. For example, the isolation container may be constructed of glass, plastic, or some other transparent or translucent and chemically inert material. Isolation containers may comprise tubes formed of any transparent or semi-transparent, flexible material (organic and inorganic), such as polystyrene, polycarbonate, styrene-butadiene-styrene, styrene/butadiene copolymer, etc., and the like. Isolation containers will typically be sterile and comprise a means for vacuum sealing.

As used herein, the term “physical barrier” refers to a substance having a density between that of red blood cells and other non-red blood cell blood components, to effectively separate and isolate red blood cells from other non-red blood cell blood components. In some embodiments, a physical barrier may comprise a relative density (i.e., specific gravity) ranging from about 1.00-1.10 g/cm³. In some embodiments described herein, a physical barrier may comprise a water insoluble thixotropic gel or gel-like substance, which is chemically inert to blood constituents and acts as density medium to separate and isolate red blood cells from other non-red blood cell blood components. In some embodiments, thixotropic gels may be formulated from a dimethyl polysiloxane and a precipitated methylated silica in which the methylation renders the material hydrophobic. In some embodiments, thixotropic gels may be formulated from a mixture of a specific hydrocarbon resin such as poly-alpha-pinene with a chlorinated hydrocarbon that forms a dual resin component appropriate for centrifugal separation. In some embodiments, the thixotropic gel preferably has a specific gravity of between about 1.055 to about 1.080 g/cm³ and is optimally formed to have a specific gravity of about 1.077 g/cm³. Exemplary methods for separating cellular components of blood using thixotropic gels are described in U.S. Pat. No. 4,957,638, which is incorporated by reference herein in its entirety.

Disclosed thixotropic gels will preferably be characterized as compositionally stable compositions, and physically stable in the absence of any substantial application of centrifugal force. Moreover, it is appropriate for the gel to form a strong cohesive barrier between the components desired to be separated, that the gel be chemically inert, and have a density which is intermediate that of the blood phases to be separated. Compositionally stable compositions mean that the components are ingredients of the separating gel which will not separate under normal storage and/or use. The composition is typically an oil, or an oil-like composition compounded with an inert filler. However, the composition should be stable enough that the oil or oil-like material does not bleed or separate from the inert filler dispersed therein. Physically stable barrier materials will not move or change shape except when subjected to the application of centrifugal force. By chemically inert, it is meant that the physical barrier material should not be chemically reactive with the blood sample being separated, its constituents or reagents commonly employed in blood separation.

In other embodiments, the physical barrier may comprise a polymer disc (i.e., “buoy”) or valve to separate and isolate red blood cells from other non-red blood cell blood components. Exemplary buoy fractionation systems are described in U.S. Pat. No. 7,992,725, which is incorporated by reference herein in its entirety.

As used herein, the terms “particles” or “plurality of particles” refer to a substance having a relative density (i.e., specific gravity) that is less than a density of a physical barrier as described herein, where the substance is configured to generate shear stress on non-red blood cell blood components (e.g., platelets and other cells). In some embodiments, the particles may comprise beads made up of polypropylene, zirconium, a combination thereof, or any other suitable material for generating shear stress on cells. In some embodiments, the plurality of particles may comprise particles having uniform or varied shapes and dimensions. For example, the plurality of particles may comprise beads having a uniform spherical shape, particles having varied and irregular dimensions, or a combination thereof.

In some embodiments, the plurality of particles may comprise a total number of particles ranging from about 1-100 particles inside of a single isolation container as described herein, depending on the particular size and density of the particles. For example, the plurality of particles may comprise a total number of particles ranging from about 1-5, 1-10, 1-20, 1-25, 1-30, 1-40, 1-50, 1-60, 1-70, 1-75, 1-80, 1-90, 1-95, 5-10, 5-20, 5-25, 5-30, 5-40, 5-50, 5-60, 5-70, 5-75, 5-80, 5-90, 5-95, 5-100, 10-20, 10-25, 10-30, 10-40, 10-50, 10-60, 10-70, 10-75, 10-80, 10-90, 10-95, 10-100, 20-25, 20-30, 20-40, 20-50, 20-60, 20-70, 20-75, 20-80, 20-90, 20-95, 20-100, 25-30, 25-40, 25-50, 25-60, 25-70, 25-75, 25-80, 25-90, 25-95, 25-100, 30-40, 30-50, 30-60, 30-70, 30-75, 30-80, 30-90, 30-95, 30-100, 40-50, 40-60, 40-70, 40-75, 40-80, 40-90, 40-95, 40-100, 50-60, 50-70, 50-75, 50-80, 50-90, 50-95, 50-100, 60-70, 60-75, 60-80, 60-90, 60-95, 60-100, 70-75, 70-80, 70-90, 70-95, 70-100, 75-80, 75-90, 75-95, 75-100, 80-90, 80-95, 80-100, 90-95, 90-100, or 95-100 particles inside of a single isolation container. In some embodiments, the plurality of particles may comprise a total number of particles inside of a single isolation container that is greater than 100 particles, including, but not limited to, up to about 110, 125, 150, 175, or 200 particles, or even greater depending on the particular size and density of the particles. In some embodiments, the plurality of particles may comprise particles having uniform or varied diameter sizes ranging from about 0.5-10 mm. For example, the plurality of particles may comprise particles having uniform or varied diameter sizes ranging from about 0.5-0.75, 0.5-0.8, 0.5-0.9, 0.5-1.0, 0.5-1.5, 0.5-2.0, 0.5-2.5, 0.5-3.0, 0.5-3.5, 0.5-4.0, 0.5-4.5, 0.5-5.0, 0.5-5.5, 0.5-6.0, 0.5-6.5, 0.5-7.0, 0.5-7.5, 0.5-8.0, 0.5-8.5, 0.5-9.0, 0.5-9.5, 0.75-0.8, 0.75-0.9, 0.75-1.0, 0.75-1.5, 0.75-2.0, 0.75-2.5, 0.75-3.0, 0.75-3.5, 0.75-4.0, 0.75-4.5, 0.75-5.0, 0.75-5.5, 0.75-6.0, 0.75-6.5, 0.75-7.0, 0.75-7.5, 0.75-8.0, 0.75-8.5, 0.75-9.0, 0.75-9.5, 0.75-10, 0.8-0.9, 0.8-1.0, 0.8-1.5, 0.8-2.0, 0.8-2.5, 0.8-3.0, 0.8-3.5, 0.8-4.0, 0.8-4.5, 0.8-5.0, 0.8-5.5, 0.8-6.0, 0.8-6.5, 0.8-7.0, 0.8-7.5, 0.8-8.0, 0.8-8.5, 0.8-9.0, 0.8-9.5, 0.8-10, 0.9-1.0, 0.9-1.5, 0.9-2.0, 0.9-2.5, 0.9-3.0, 0.9-3.5, 0.9-4.0, 0.9-4.5, 0.9-5.0, 0.9-5.5, 0.9-6.0, 0.9-6.5, 0.9-7.0, 0.9-7.5, 0.9-8.0, 0.9-8.5, 0.9-9.0, 0.9-9.5, 0.9-10, 1.0-1.5, 1.0-2.0, 1.0-2.5, 1.0-3.0, 1.0-3.5, 1.0-4.0, 1.0-4.5, 1.0-5.0, 1.0-5.5, 1.0-6.0, 1.0-6.5, 1.0-7.0, 1.0-7.5, 1.0-8.0, 1.0-8.5, 1.0-9.0, 1.0-9.5, 1.0-10, 1.5-2.0, 1.5-2.5, 1.5-3.0, 1.5-3.5, 1.5-4.0, 1.5-4.5, 1.5-5.0, 1.5-5.5, 1.5-6.0, 1.5-6.5, 1.5-7.0, 1.5-7.5, 1.5-8.0, 1.5-8.5, 1.5-9.0, 1.5-9.5, 1.5-10, 2.0-2.5, 2.0-3.0, 2.0-3.5, 2.0-4.0, 2.0-4.5, 2.0-5.0, 2.0-5.5, 2.0-6.0, 2.0-6.5, 2.0-7.0, 2.0-7.5, 2.0-8.0, 2.0-8.5, 2.0-9.0, 2.0-9.5, 2.0-10, 2.5-3.0, 2.5-3.5, 2.5-4.0, 2.5-4.5, 2.5-5.0, 2.5-5.5, 2.5-6.0, 2.5-6.5, 2.5-7.0, 2.5-7.5, 2.5-8.0, 2.5-8.5, 2.5-9.0, 2.5-9.5, 2.5-10, 3.0-3.5, 3.0-4.0, 3.0-4.5, 3.0-5.0, 3.0-5.5, 3.0-6.0, 3.0-6.5, 3.0-7.0, 3.0-7.5, 3.0-8.0, 3.0-8.5, 3.0-9.0, 3.0-9.5, 3.0-10, 3.5-4.0, 3.5-4.5, 3.5-5.0, 3.5-5.5, 3.5-6.0, 3.5-6.5, 3.5-7.0, 3.5-7.5, 3.5-8.0, 3.5-8.5, 3.5-9.0, 3.5-9.5, 3.5-10, 4.0-4.5, 4.0-5.0, 4.0-5.5, 4.0-6.0, 4.0-6.5, 4.0-7.0, 4.0-7.5, 4.0-8.0, 4.0-8.5, 4.0-9.0, 4.0-9.5, 4.0-10, 4.5-5.0, 4.5-5.5, 4.5-6.0, 4.5-6.5, 4.5-7.0, 4.5-7.5, 4.5-8.0, 4.5-8.5, 4.5-9.0, 4.5-9.5, 4.5-10, 5.0-5.5, 5.0-6.0, 5.0-6.5, 5.0-7.0, 5.0-7.5, 5.0-8.0, 5.0-8.5, 5.0-9.0, 5.0-9.5, 5.0-10, 5.5-6.0, 5.5-6.5, 5.5-7.0, 5.5-7.5, 5.5-8.0, 5.5-8.5, 5.5-9.0, 5.5-9.5, 5.5-10, 6.0-6.5, 6.0-7.0, 6.0-7.5, 6.0-8.0, 6.0-8.5, 6.0-9.0, 6.0-9.5, 6.0-10, 6.5-7.0, 6.5-7.5, 6.5-8.0, 6.5-8.5, 6.5-9.0, 6.5-9.5, 6.5-10, 7.0-7.5, 7.0-8.0, 7.0-8.5, 7.0-9.0, 7.0-9.5, 7.0-10, 7.5-8.0, 7.5-8.5, 7.5-9.0, 7.5-9.5, 7.5-10, 8.0-8.5, 8.0-9.0, 8.0-9.5, 8.0-10, 8.5-9.0, 8.5-9.5, 8.5-10, 9.0-9.5, 9.0-10, or 9.5-10 mm. In some embodiments, the plurality of particles may comprise a density ranging from about 0.95-1.05 g/cm³.

As used herein, the terms “agitate,” “agitation,” or “agitating” refer to an act of providing a constant or irregular mechanical stimulation or forceful motion to an object. In some embodiments, agitating may comprise the use of shaking, vortexing, or other common techniques well-known to one of skill in the art. In some embodiments, agitating may comprise the use of an agitator device well-known in the art to induce agitation. In some embodiments, isolation containers may be agitated at any angle ranging from about 30° to about 60° relative to a below surface to generate shear stress on one or more non-red blood cell blood components. In preferred embodiments, agitating the isolation container comprises agitation at an angle of about 45° relative to a below surface to generate shear stress most effectively.

### Methods of Isolation

Described herein are methods for isolating exosomes or extracellular vesicles from a blood sample or a platelet-rich plasma (PRP) sample. In some embodiments, the methods may comprise one or more centrifugation steps of an isolation container comprising a blood sample to separate non-red blood cell blood components from red blood cells and generate a PRP sample. In some embodiments, the methods may comprise one or more agitation steps of an isolation container comprising a blood sample to generate shear stress on non-red blood cell blood components and release exosomes or extracellular vesicles from a PRP sample. In some embodiments, the methods may further comprise one or more filtering steps of an exosome-rich PRP (ER-PRP) sample to remove platelets and/or white blood cells.

Centrifugation steps may be performed, for example, at about 100-7000×g for about 5-60 minutes. In some embodiments, centrifugation is performed at about 500-2500×g for about 10-30 minutes. In non-limiting exemplary embodiments, a blood sample is centrifuged at a specific speed, centrifugal force, and time sufficient to separate blood components (typically 4000-7000 RPM in a clinical centrifuge; e.g., 1000-1500×g for 10-20 min). Centrifugation is typically performed at about 4° C. to about 20° C. Any suitable laboratory or clinical centrifuge may be employed to conduct centrifugation steps. In some embodiments, a single centrifugation step is performed to separate non-red blood cell blood components from red blood cells and generate a PRP sample. In other embodiments, multiple centrifugation steps are performed to separate non-red blood cell blood components from red blood cells and generate a PRP sample.

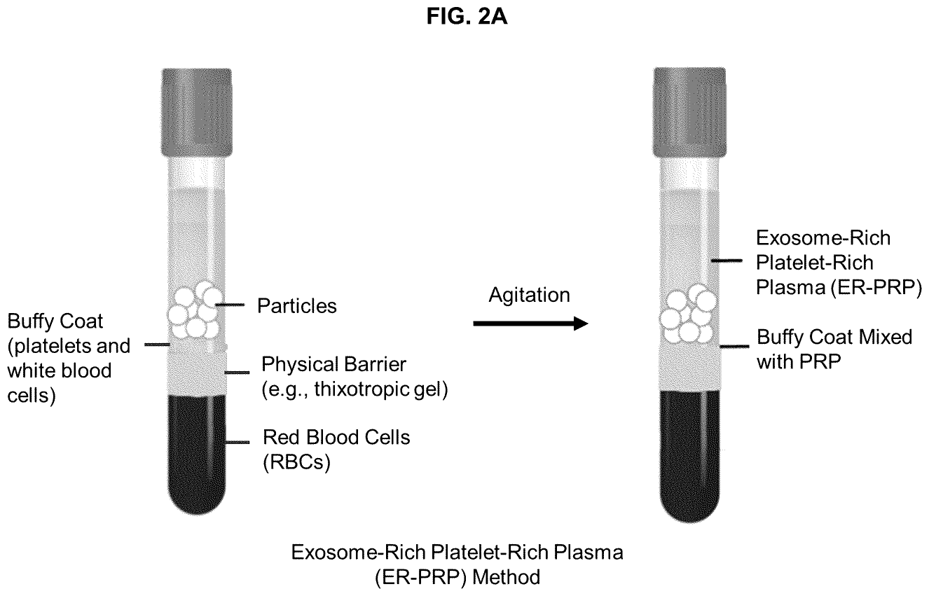

Pure preparations of autologous, platelet-rich plasma (PRP)-derived exosomes can be isolated by collecting whole blood, centrifuging the blood, and using particles (e.g., beads) to provide mechanical shear force upon agitation after centrifugal separation. By shaking a PRP in the presence of beads, shear force allows for the release of the extracellular vesicles and exosomes from platelets and other cells within the buffy coat without immediate activation of the clotting cascade. This results in a non-clotted preparation of PRP enriched with significantly greater quantities of free extracellular vesicles and exosomes, called exosome-rich platelet-rich plasma (ER-PRP) (FIG. 2A). Additionally, this ER-PRP can then be further purified using, for example, a hypodermic needle connected to a 0.1-μm sterile filter and syringe. As the exosome-rich platelet-rich plasma is drawn into the syringe, the filter removes particles larger than extracellular vesicles and results in a more refined exosome-rich plasma (ERP) (FIG. 2B).

The methods described herein advantageously provide a means to rapidly obtain mammalian and non-mammalian exosomes with high purity which are useful diagnostically and/or therapeutically. In some embodiments, the methods yield exosomes which exhibit a high degree of purity, for example, at least about 50% pure, and preferably, at least about 60%, 70%, 80%, 90% or 95% or greater pure. Preferably, isolated exosomes are “essentially free of” cellular debris, apoptotic bodies, and microvesicles having a diameter less than about 20 nm or greater than about 150 nm, and preferably less than 40 nm or greater than 120 nm, and are biologically intact, e.g., not clumped or in aggregate form, and not sheared, leaky, or otherwise damaged. Exosomes isolated according to the methods described herein exhibit a degree of stability, that may be evidenced by the zeta potential of an isolated mixture/solution of such exosomes, for example, a zeta potential of at least a magnitude of ±10 mV, e.g. ≤−10 or ≥±10, and preferably, a magnitude of at least 20 mV, 30 mV, 40 mV, 50 mV, 60 mV, 70 mV, 80 mV, or greater. The term “zeta potential” refers to the electrokinetic potential of a colloidal dispersion, and the magnitude of the zeta potential indicates the degree of electrostatic repulsion between adjacent, similarly charged particles (exosomes) in a dispersion. For isolated exosomes, generally the higher the magnitude of the zeta potential, the greater the stability of the exosomes.

In some embodiments, isolated exosomes or extracellular vesicles may be stored for later use or analysis, for example, in cold storage at 4° C., in frozen form, or in lyophilized form, prepared using well-established protocols. The isolated exosomes or extracellular vesicles may be stored in any physiological acceptable carrier or buffer, optionally including cryogenic stability and/or vitrification agents (e.g., DMSO, glycerol, trehalose, polyhydroxylated alcohols (e.g., methoxylated glycerol, propylene glycol), and the like).

### Systems for Isolation

Described herein are systems for isolating exosomes or extracellular vesicles from a blood sample or a platelet-rich plasma (PRP) sample. In some embodiments, the systems may comprise one or more isolation containers comprising one or more physical barriers configured to separate non-red blood cell blood components from red blood cells in a blood sample. In some embodiments, the systems may comprise a plurality of particles having a particle density that is less than a density of a physical barrier, the plurality of particles being configured to generate shear stress on non-red blood cell blood components. In some embodiments, the systems may comprise one or more anticoagulants. In one aspect, the systems may further comprise a centrifuge to separate non-red blood cell blood components from red blood cells. In another aspect, the systems may further comprise an agitator to generate shear stress on non-red blood cell blood components. Another embodiment described herein is the use of the systems described herein for the isolation of exosomes or extracellular vesicles.

One embodiment described herein is a method for isolating exosomes or extracellular vesicles from a blood sample, the method comprising: adding a blood sample to an isolation container comprising: a physical barrier configured to separate non-red blood cell blood components from red blood cells; a plurality of particles having a particle density that is less than a density of the physical barrier, the plurality of particles being configured to generate shear stress on non-red blood cell blood components; and an anticoagulant; centrifuging the isolation container to separate non-red blood cell blood components from red blood cells, thereby generating a platelet-rich plasma (PRP); and agitating the isolation container to generate shear stress on one or more non-red blood cell blood components and release exosomes or extracellular vesicles from the PRP, thereby generating an exosome-rich PRP (ER-PRP). In one aspect, the method, further comprises filtering the ER-PRP to remove platelets and generate an exosome-rich plasma (ERP). In another aspect, filtering comprises the use of a filter having pores ranging from about 0.1 μm to about 5 μm in size. In another aspect, the method, further comprises filtering the ER-PRP to remove white blood cells. In another aspect, filtering comprises the use of a filter having pores ranging from about 0.1 μm to about 20 μm in size. In another aspect, the isolation container comprises a glass tube or a plastic tube. In another aspect, the physical barrier comprises a valve, a polymer disc or buoy, or a thixotropic gel. In another aspect, the physical barrier comprises a density ranging from about 1.05 g/cm³ to about 1.10 g/cm³. In another aspect, the plurality of particles comprises a plurality of beads comprised of polypropylene, zirconium, or a combination thereof. In another aspect, the plurality of particles comprises a density ranging from about 0.95 g/cm³ to about 1.05 g/cm³. In another aspect, the anticoagulant comprises anticoagulant citrate dextrose solution A (ACDA), calcium citrate, or a combination thereof. In another aspect, centrifuging the isolation container comprises centrifugation at about 100-7000×g for about 5-60 minutes to separate non-red blood cell blood components from red blood cells. In another aspect, centrifuging the isolation container comprises centrifugation at about 1000-1500×g for about 10-20 minutes to separate non-red blood cell blood components from red blood cells. In another aspect, agitating the isolation container comprises agitation at an angle of about 30° to about 60° to generate shear stress on the one or more non-red blood cell blood components. In another aspect, agitating the isolation container comprises agitation at an angle of about 45° to generate shear stress on the one or more non-red blood cell blood components.

Another embodiment described herein is exosomes or extracellular vesicles isolated using the methods described herein.

Another embodiment described herein is a method for isolating exosomes or extracellular vesicles from a platelet-rich plasma (PRP) sample, the method comprising: adding a PRP sample to an isolation container comprising: a plurality of particles configured to generate shear stress on non-red blood cell blood components; and an anticoagulant; and agitating the isolation container to generate shear stress on one or more non-red blood cell blood components and release exosomes or extracellular vesicles from the PRP sample, thereby generating an exosome-rich PRP (ER-PRP). In one aspect, the method, further comprises filtering the ER-PRP to remove platelets and generate an exosome-rich plasma (ERP). In another aspect, filtering comprises the use of a filter having pores ranging from about 0.1 μm to about 5 μm in size. In another aspect, the method, further comprises filtering the ER-PRP to remove white blood cells. In another aspect, filtering comprises the use of a filter having pores ranging from about 0.1 μm to about 20 μm in size.

Another embodiment described herein is exosomes or extracellular vesicles isolated using the methods described herein.

Another embodiment described herein is a system for isolating exosomes or extracellular vesicles from a blood sample, the system comprising: an isolation container comprising a physical barrier configured to separate non-red blood cell blood components from red blood cells in a blood sample; a plurality of particles having a particle density that is less than a density of the physical barrier, the plurality of particles being configured to generate shear stress on non-red blood cell blood components; and an anticoagulant. In one aspect, the system further comprises a centrifuge to separate non-red blood cell blood components from red blood cells. In another aspect, the system further comprises an agitator to generate shear stress on non-red blood cell blood components.

Another embodiment described herein is exosomes or extracellular vesicles isolated using the systems described herein.

Another embodiment described herein is the use of the systems described herein for the isolation of exosomes or extracellular vesicles.

Another embodiment described herein is a kit for isolating exosomes or extracellular vesicles from a blood sample, the kit comprising: an isolation container comprising: a physical barrier configured to separate non-red blood cell blood components from red blood cells in a blood sample; a plurality of particles having a particle density that is less than a density of the physical barrier, the plurality of particles being configured to generate shear stress on non-red blood cell blood components; and an anticoagulant; optionally, one or more of a filter, a hypodermic needle, or a syringe; optionally, buffers and receptacles; and optionally, one or more of packaging or instructions for use.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for isolating exosomes or extracellular vesicles from a blood sample, the method comprising:
   (a) adding a blood sample to an isolation container comprising:
      a physical barrier configured to separate non-red blood cell blood components from red blood cells;
      a plurality of particles having a particle density that is less than a density of the physical barrier, the plurality of particles being configured to generate shear stress on non-red blood cell blood components; and
      an anticoagulant;
   (b) centrifuging the isolation container to separate non-red blood cell blood components from red blood cells, thereby generating a platelet-rich plasma (PRP); and
   (c) agitating the isolation container to generate shear stress on one or more non-red blood cell blood components and release exosomes or extracellular vesicles from the PRP, thereby generating an exosome-rich PRP (ER-PRP).
Clause 2. The method of clause 1, further comprising filtering the ER-PRP to remove platelets and generate an exosome-rich plasma (ERP).
Clause 3. The method of clause 1 or 2, wherein filtering comprises the use of a filter having pores ranging from about 0.1 μm to about 5 μm in size.
Clause 4. The method of any one of clauses 1-3, further comprising filtering the ER-PRP to remove white blood cells.
Clause 5. The method of any one of clauses 1-4, wherein filtering comprises the use of a filter having pores ranging from about 0.1 μm to about 20 μm in size.
Clause 6. The method of any one of clauses 1-5, wherein the isolation container comprises a glass tube or a plastic tube.
Clause 7. The method of any one of clauses 1-6, wherein the physical barrier comprises a valve, a polymer disc or buoy, or a thixotropic gel.
Clause 8. The method of any one of clauses 1-7, wherein the physical barrier comprises a density ranging from about 1.05 g/cm³ to about 1.10 g/cm³.
Clause 9. The method of any one of clauses 1-8, wherein the plurality of particles comprises a plurality of beads comprised of polypropylene, zirconium, or a combination thereof.
Clause 10. The method of any one of clauses 1-9, wherein the plurality of particles comprises a density ranging from about 0.95 g/cm³ to about 1.05 g/cm³.
Clause 11. The method of any one of clauses 1-10, wherein the anticoagulant comprises anticoagulant citrate dextrose solution A (ACDA), calcium citrate, or a combination thereof.
Clause 12. The method of any one of clauses 1-11, wherein centrifuging the isolation container comprises centrifugation at about 100-7000×g for about 5-60 minutes to separate non-red blood cell blood components from red blood cells.
Clause 13. The method of any one of clauses 1-12, wherein centrifuging the isolation container comprises centrifugation at about 1000-1500×g for about 10-20 minutes to separate non-red blood cell blood components from red blood cells.
Clause 14. The method of any one of clauses 1-13, wherein agitating the isolation container comprises agitation at an angle of about 30° to about 60° to generate shear stress on the one or more non-red blood cell blood components.
Clause 15. The method of any one of clauses 1-14, wherein agitating the isolation container comprises agitation at an angle of about 45° to generate shear stress on the one or more non-red blood cell blood components.
Clause 16. Exosomes or extracellular vesicles isolated using the method of any one of clauses 1-15.
Clause 17. A method for isolating exosomes or extracellular vesicles from a platelet-rich plasma (PRP) sample, the method comprising:
   (a) adding a PRP sample to an isolation container comprising:
      a plurality of particles configured to generate shear stress on non-red blood cell blood components; and
      an anticoagulant; and
   (b) agitating the isolation container to generate shear stress on one or more non-red blood cell blood components and release exosomes or extracellular vesicles from the PRP sample, thereby generating an exosome-rich PRP (ER-PRP).
Clause 18. The method of clause 17, further comprising filtering the ER-PRP to remove platelets and generate an exosome-rich plasma (ERP).
Clause 19. The method of clause 17 or 18, wherein filtering comprises the use of a filter having pores ranging from about 0.1 μm to about 5 μm in size.
Clause 20. The method of any one of clauses 17-19, further comprising filtering the ER-PRP to remove white blood cells.
Clause 21. The method of any one of clauses 17-20, wherein filtering comprises the use of a filter having pores ranging from about 0.1 μm to about 20 μm in size.
Clause 22. Exosomes or extracellular vesicles isolated using the method of any one of clauses 17-21.
Clause 23. A system for isolating exosomes or extracellular vesicles from a blood sample, the system comprising:
   an isolation container comprising a physical barrier configured to separate non-red blood cell blood components from red blood cells in a blood sample;
   a plurality of particles having a particle density that is less than a density of the physical barrier, the plurality of particles being configured to generate shear stress on non-red blood cell blood components; and an anticoagulant.
Clause 24. The system of clause 23, further comprising a centrifuge to separate non-red blood cell blood components from red blood cells.
Clause 25. The system of clause 23 or 24, further comprising an agitator to generate shear stress on non-red blood cell blood components.
Clause 26. Exosomes or extracellular vesicles isolated using the system of any one of clauses 23-25.
Clause 27. Use of the system of any one of clauses 23-25 for the isolation of exosomes or extracellular vesicles.
Clause 28. A kit for isolating exosomes or extracellular vesicles from a blood sample, the kit comprising:
   an isolation container comprising:
      a physical barrier configured to separate non-red blood cell blood components from red blood cells in a blood sample;
      a plurality of particles having a particle density that is less than a density of the physical barrier, the plurality of particles being configured to generate shear stress on non-red blood cell blood components; and
      an anticoagulant;
   optionally, one or more of a filter, a hypodermic needle, or a syringe;
   optionally, buffers and receptacles; and
   optionally, one or more of packaging or instructions for use.

### EXAMPLES

### Example 1

Whole blood was collected from a subject using a sterile, vacuum-sealed blood isolation tube (e.g., 10 mL to 100 mL), which includes an internal physical barrier that has a density between red blood cells and the other non-red blood cell blood components (i.e., leukocytes, platelets, exosomes), such as a thixotropic gel, polymer disc (i.e., “buoy”), or valve. The collection container also contained beads or small particle fragments of a biocompatible substance such as polypropylene that has a density less than the physical barrier. The container also contained an anticoagulant, such as Anticoagulant Citrate Dextrose A Solution, USP (ACD-A, anhydrous citric acid, dextrose monohydrate, and trisodium citrate dihydrate solution) or calcium citrate.

After collection, the blood was centrifuged at a specific speed, centrifugal force, and time sufficient to separate the blood components (typically 4000-7000 RPM in a clinical centrifuge; e.g., 1000-1500×g for 10-20 min).

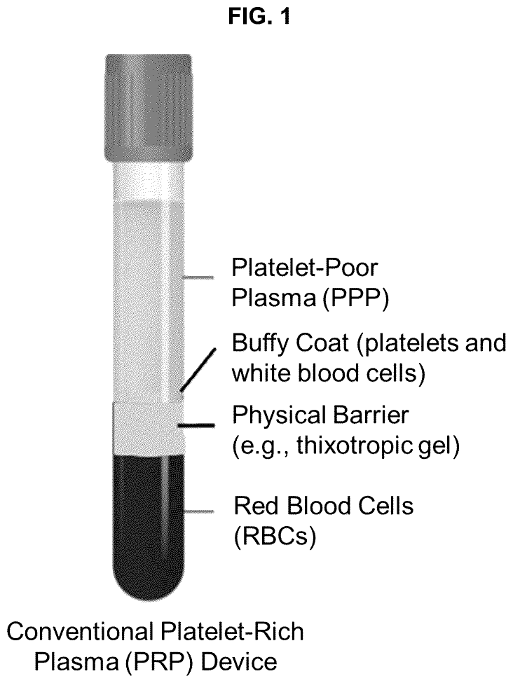

After centrifugation, the blood components separated into three distinct layers: platelet-poor plasma (PPP) in the upper phase; the buffy coat, which is a thin layer of leukocytes (white blood cells) and platelets; and erythrocytes (red blood cells) at the bottom of the centrifuge tube. If a thixotropic gel is used, the buffy coat collects on the surface of the gel (e.g., see FIG. 1).

To enrich the plasma sample for exosomes and extracellular vesicles, the tube was shaken or vortexed (i.e., agitated) at a ˜45°-angle permitting the beads to shear the platelets and other cells, thereby releasing the exosomes and extracellular vesicles. This resulted in an exosome-rich platelet-rich plasma (ER-PRP) (FIG. 2A).

After shearing, a syringe with a sterile 0.1-μm disc filter and hypodermic needle was used to decant and filter the ER-PRP to generate an exosome-rich plasma (ERP) (FIG. 2B). This was be dispensed into a sterile tube for further processing, analysis, or storage.

### Example 2

Exosomes were isolated using two methods, with and without shearing beads. In the method without beads, exosomes were isolated using polyethylene terephthalate (PET) tubes with serum separation gel, anti-coagulant, and a rubber stopper. The exosomes isolated using this method were referred to as “PRP-Shake.” In the method with beads, exosomes were isolated using PET tubes with serum separation gel, anti-coagulant, beads to break open cells, and a rubber stopper. T The exosomes isolated using this method were referred to as “PRP-Beads.” The exosomes present in blood plasma are referred to as “Plasma Control.” The exosomes isolated from these three sample types were analyzed as follows.

### Physiochemical Characterization

Serum isolated using the different methods was centrifuged at 7000×g to remove cells and cell debris. The samples were then diluted 1:50000 in ultrapure deionized H₂O. The diluted samples were characterized using Nanoparticle Tracking Analysis (Particle Metrix Zetaview®) for their size, and concentration. The experiments were performed using five different subjects and the results are presented as an average range between the subjects.

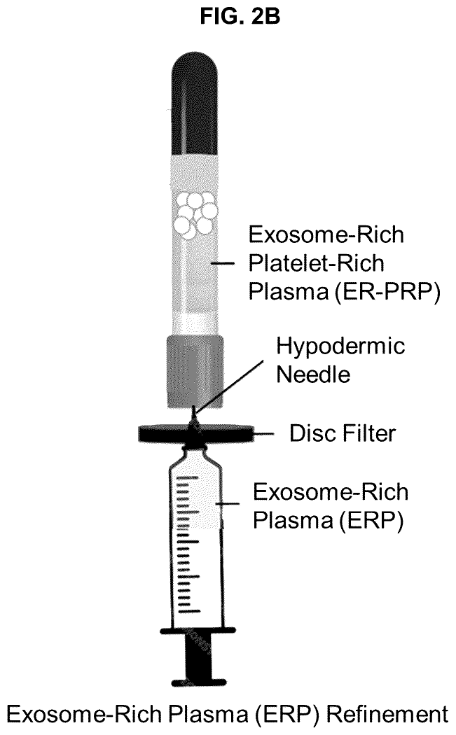

Samples from blood plasma had an average size of 115.8 nm with a standard deviation of 2.37 nm among the subjects, the PRP Beads had an average size of 123 nm with a standard deviation of 2.99 nm between the subjects and the PRP Shake had an average size of 122.08 nm with a standard deviation of 3.38 nm among the subjects (Table 2, FIG. 3). This demonstrated that the measured particles are exosomes (30-200 nm) and have little variation between subjects. There was also a close tolerance within the groups where the range was less than 70 nm deviation (Table 1). Interestingly a small size variation was observed for Plasma Control exosomes compared with the other samples. Though not a significant change, the exosomes isolated from plasma control were smaller in size and had a wider size distribution (Table 2) compared to that of exosomes isolated from the devices.

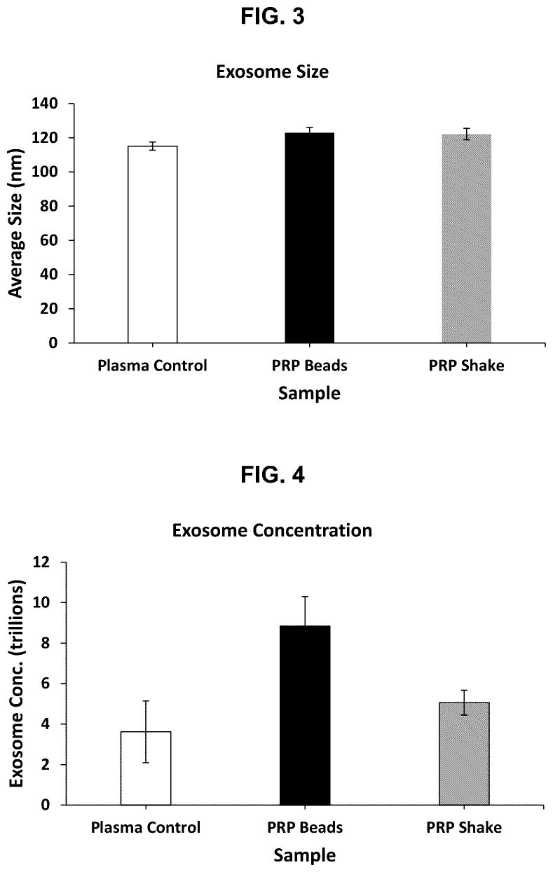

Although there was no significant difference in the exosome sizes among the samples, the concentration measurement highlights the significance of beads in exosome release. The average exosome concentration obtained from the Plasma Control, PRP Beads, and PRP shake were 3.62, 8.84, and 5.06 trillion, respectively (Table 2, FIG. 4). Isolation of exosomes using the serum separation gel both with and without beads had greater exosome release. PRP without beads had 1.4× and PRP with beads had 2.44× greater release compared to that of plasma exosomes (Table 2). A significant increase (1.74×) was observed for exosome released using beads as compared to exosomes released without beads. This was attributed to the beads stressing and shearing the platelets and white blood cells facilitating the release of exosomes. While the number of exosomes varied for each subject, the ratio between the groups for a subject had similar trends and the beads increased the exosome concentration.

TABLE 1
Extracted Exosome Size Measured
by Nanoparticle Tracking Analysis

| | | | | Concentration |
|---|---|---|---|---|
| Subject | Sample Prep | Size (nm) | Std Dev | (trillion) |
| Subject 1 | Plasma Control | 112.4 | 49.1 | 4.9 |
| | PRP Beads | 124 | 53.2 | 7.3 |
| | PRP Shake | 126.7 | 48.6 | 4.1 |
| Subject 2 | Plasma Control | 117 | 46.7 | 4.9 |
| | PRP Beads | 122 | 49.2 | 9.3 |
| | PRP Shake | 120.8 | 45.2 | 5 |
| Subject 3 | Plasma Control | 118.7 | 69.9 | 1.7 |
| | PRP Beads | 118.6 | 63.6 | 9.5 |
| | PRP Shake | 116.6 | 60.7 | 5.9 |
| Subject 4 | Plasma Control | 113.5 | 62.9 | 4.8 |
| | PRP Beads | 127.8 | 68.1 | 11 |
| | PRP Shake | 124.1 | 65.5 | 4.8 |
| Subject 5 | Plasma Control | 113.8 | 71.6 | 1.8 |
| | PRP Beads | 122.6 | 61.6 | 7.1 |
| | PRP Shake | 122.2 | 60.2 | 5.5 |

TABLE 2
Average Exosome Size and Number
Measured By Nanoparticle Tracking

| Analysis | Average | | Concentration | |
|---|---|---|---|---|
| Sample Prep. | Size (nm) | Std Dev | (trillion) | Std Dev |
| Plasma Control | 115.08 | 2.37 | 3.62 | 1.53 |
| PRP Beads | 123.00 | 2.99 | 8.84 | 1.46 |
| PRP Shake | 122.08 | 3.38 | 5.06 | 0.62 |

### Example 3

### Genomic and Proteomic Analysis

Gene ontology (GO-Gene Ontology Consortium, 2000) enrichment analysis is a set of the internationally standardized classification system of gene function description that attempts to identify GO terms that are significantly associated with differentially expressed protein coding genes. GO molecules are divided into three main categories: (1) Cellular Component: used to describe the subcellular structure, location and macromolecular complexes, such as nucleoli, telomere and recognition of the initial complex; (2) Molecular Function: used to describe the gene, gene products, individual functions, such as carbohydrate binding or ATP hydrolase activity; (3) Biological Process: used to describe the products encoded by genes involved in biological processes, such as mitosis or purine metabolism.

### Genomic Analysis

Serum was isolated from samples prepared using the PRP Shake and PRP Beads methods as described above in Example 2. The samples were analysed for their RNA contents by genomic analysis. The experiments were performed by CD Genomics (New York, USA). The analysis was a pilot study with one subject. Data is shown in Table 3.

TABLE 3
Gene Ontology Functional Analysis

| | | | | |
|---|---|---|---|---|
| | | | | −log10 |
| Ontology Term | P-value | Count | Percentage | (p-value) |

Biological Process

| | | | | |
|---|---|---|---|---|
| Modulation of Chemical | 5.45 × 10⁻¹³ | 307 | 3.12 | 12.264 |
| Synaptic Transmission | | | | |
| Regulation of Trans- | 8.39 × 10⁻¹³ | 307 | 3.12 | 12.076 |
| Synaptic Signaling | | | | |
| Regulation of Neuron | 3.94 × 10⁻¹² | 303 | 3.08 | 11.405 |
| Projection Development | | | | |
| Cell Junction Assembly | 2.08 × 10⁻¹¹ | 291 | 2.96 | 10.681 |
| Synapse Organization | 2.38 × 10⁻¹¹ | 292 | 2.97 | 10.624 |
| Axon Development | 1.13 × 10⁻¹⁰ | 324 | 3.29 | 9.948 |
| Axonogenesis | 1.30 × 10⁻¹⁰ | 293 | 2.98 | 9.884 |
| Neurotransmitter | 2.22 × 10⁻¹⁰ | 155 | 1.58 | 9.653 |
| Transport | | | | |
| Ras Protein Signal | 3.00 × 10⁻¹⁰ | 241 | 2.45 | 9.523 |
| Transduction | | | | |
| Regulation of | 3.16 × 10⁻¹⁰ | 159 | 1.62 | 9.500 |
| Neurotransmitter Levels | | | | |

Molecular Function

| | | | | |
|---|---|---|---|---|
| Protein Serine/Threonine | 2.29 × 10⁻⁸ | 290 | 2.89 | 7.640 |
| Kinase Activity | | | | |
| Molecular Adaptor | 3.17 × 10⁻⁸ | 327 | 3.25 | 7.499 |
| Activity | | | | |
| GTPase Regulator | 1.21 × 10⁻⁷ | 322 | 3.20 | 6.918 |
| Activity | | | | |
| Nucleoside- | 1.21 × 10⁻⁷ | 322 | 3.20 | 6.918 |
| Triphosphatase | | | | |
| Regulator Activity | | | | |
| Phosphoric Ester | 1.60 × 10⁻⁷ | 247 | 2.46 | 6.796 |
| Hydrolase Activity | | | | |
| DNA-Binding | 6.48 × 10⁻⁷ | 309 | 3.07 | 6.188 |
| Transcription | | | | |
| Activator Activity | | | | |
| Protein Serine Kinase | 7.72 × 10⁻⁷ | 243 | 2.42 | 6.113 |
| Activity DNA-Binding | | | | |
| Transcription | | | | |
| Activator Activity, Rna | 8.68 × 10⁻⁷ | 306 | 3.05 | 6.061 |
| Polymerase li-Specific | | | | |
| Protein-Macromolecule | 9.05 × 10⁻⁷ | 233 | 2.32 | 6.044 |
| Adaptor Activity | | | | |
| Small GTPase Binding | 9.80 × 10⁻⁷ | 187 | 1.86 | 6.009 |

Cellular Component

| | | | | |
|---|---|---|---|---|
| Synaptic Membrane | 1.41 × 10⁻¹⁷ | 276 | 2.68 | 16.851 |
| Neuron To Neuron | 2.66 × 10⁻¹⁶ | 256 | 2.49 | 15.575 |
| Synapse | | | | |
| Asymmetric Synapse | 4.44 × 10⁻¹⁵ | 238 | 2.31 | 14.353 |
| Postsynaptic Density | 3.55 × 10⁻¹⁴ | 232 | 2.25 | 13.450 |
| Postsynaptic | 2.01 × 10⁻¹³ | 243 | 2.36 | 12.696 |
| Specialization | | | | |
| Membrane Microdomain | 3.87 × 10⁻¹³ | 233 | 2.26 | 12.412 |
| Membrane Raft | 5.38 × 10⁻¹³ | 232 | 2.25 | 12.269 |
| Neuronal Cell Body | 1.36 × 10⁻¹² | 334 | 3.24 | 11.867 |
| Neuron Spine | 7.37 × 10⁻¹² | 135 | 1.31 | 11.133 |
| Dendritic Spine | 1.10 × 10⁻¹¹ | 134 | 1.30 | 10.959 |

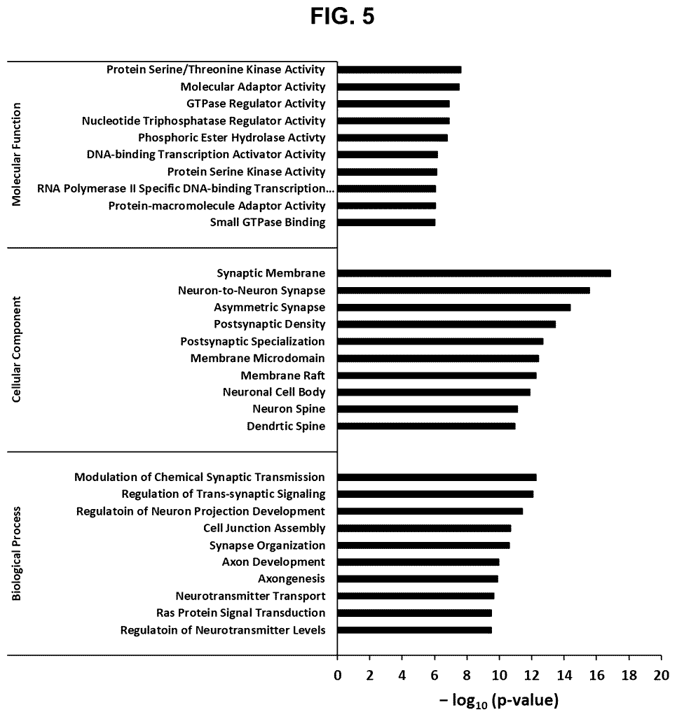

When the exosomes from PRP Beads were compared with the exosomes from PRP Shake, there was significant enrichment in all the three categories (FIG. 5). There was significant enrichment in neural signaling and neuronal development pathways with genes correlated to axonogenesis and axon development. While further analysis is needed to understand the translation of these genes, the genes are likely to have a lymphocyte origin.

### Proteomic Analysis

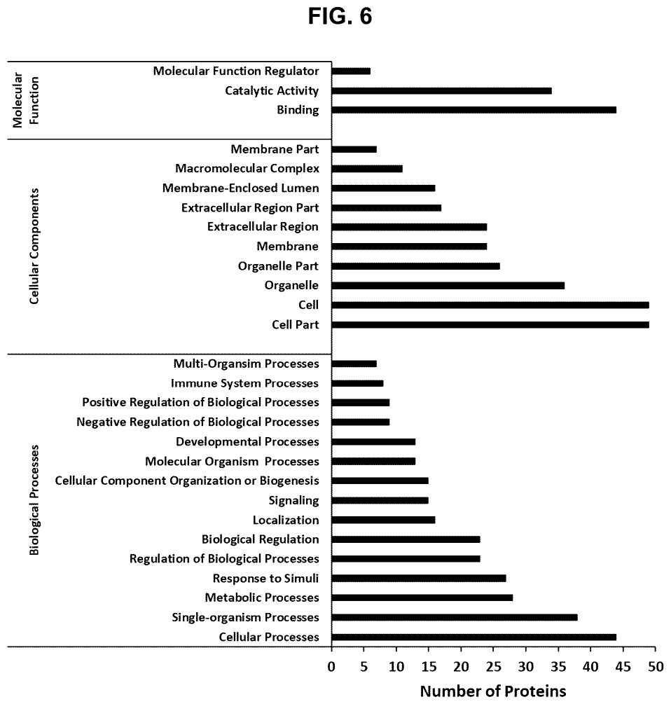

Quantitative proteomic analysis was performed to analyze the differentially expressed protein between the PRP Beads and PRP shake. Several upregulated proteins were observed in the exosomes isolated from PRP beads compared to those isolated from PRP Shake. These differentially expressed proteins were investigated by Gene Ontology functional analyses to identify the biological process, cellular components, and molecular function (FIG. 6). Data is shown in Table 4.

TABLE 4
Proteomic Analysis

| | |
|---|---|
| GO Name | Number of Proteins |

Biological Processes

| | |
|---|---|
| Cellular Processes | 44 |
| Single-organism Processes | 38 |
| Metabolic Processes | 28 |
| Response to Simuli | 27 |
| Regulation of Biological Processes | 23 |
| Biological Regulation | 23 |
| Localization | 16 |
| Signaling | 15 |
| Cellular Component Organization or Biogenesis | 15 |
| Molecular Organism Processes | 13 |
| Developmental Processes | 13 |
| Negative Regulation of Biological Processes | 9 |
| Positive Regulation of Biological Processes | 9 |
| Immune System Processes | 8 |
| Multi-Organsim Processes | 7 |
| Biological Adhesion | 6 |

Cellular Components

| | |
|---|---|
| Cell Part | 49 |
| Cell | 49 |
| Organelle | 36 |
| Organelle Part | 26 |
| Membrane | 24 |
| Extracellular Region | 24 |
| Extracellular Region Part | 17 |
| Membrane-Enclosed Lumen | 16 |
| Macromolecular Complex | 11 |
| Membrane Part | 7 |
| Synapse | 7 |

Molecular Function

| | |
|---|---|
| Binding | 44 |
| Catalytic Activity | 34 |
| Molecular Function Regulator | 6 |

Gene Ontology functional shows enrichment of proteins attributed to biological process including cellular process, single-organism process, metabolic process, biological regulation, regulation of biological process, response to stimulus. GO enrichment showed enrichment of proteins that are related to extracellular region, membrane, organelle, membrane-enclosed lumen. Proteins having molecular functions such as binding and catalytic activity were also enriched.

1. A method for isolating exosomes or extracellular vesicles from a blood sample, the method comprising:
(a) adding a blood sample to an isolation container comprising:
a physical barrier configured to separate non-red blood cell blood components from red blood cells;
a plurality of particles having a particle density that is less than a density of the physical barrier, the plurality of particles being configured to generate shear stress on non-red blood cell blood components; and
an anticoagulant;
(b) centrifuging the isolation container to separate non-red blood cell blood components from red blood cells, thereby generating a platelet-rich plasma (PRP); and
(c) agitating the isolation container to generate shear stress on one or more non-red blood cell blood components and release exosomes or extracellular vesicles from the PRP, thereby generating an exosome-rich PRP (ER-PRP).

2. The method of claim 1, further comprising filtering the ER-PRP to remove platelets and generate an exosome-rich plasma (ERP).

3. The method of claim 2, wherein filtering comprises the use of a filter having pores ranging from about 0.1 μm to about 5 μm in size.

4. The method of claim 1, further comprising filtering the ER-PRP to remove white blood cells.

5. The method of claim 4, wherein filtering comprises the use of a filter having pores ranging from about 0.1 μm to about 20 μm in size.

6. The method of claim 1, wherein the isolation container comprises a glass tube or a plastic tube.

7. The method of claim 1, wherein the physical barrier comprises a valve, a polymer disc or buoy, or a thixotropic gel.

8. The method of claim 1, wherein the physical barrier comprises a density ranging from about 1.05 g/cm³ to about 1.10 g/cm³.

9. The method of claim 1, wherein the plurality of particles comprises a plurality of beads comprised of polypropylene, zirconium, or a combination thereof.

10. The method of claim 1, wherein the plurality of particles comprises a density ranging from about 0.95 g/cm³ to about 1.05 g/cm³.

11. The method of claim 1, wherein the anticoagulant comprises anticoagulant citrate dextrose solution A (ACDA), calcium citrate, or a combination thereof.

12. The method of claim 1, wherein centrifuging the isolation container comprises centrifugation at about 100-7000×g for about 5-60 minutes to separate non-red blood cell blood components from red blood cells.

13. The method of claim 12, wherein centrifuging the isolation container comprises centrifugation at about 1000-1500×g for about 10-20 minutes to separate non-red blood cell blood components from red blood cells.

14. The method of claim 1, wherein agitating the isolation container comprises agitation at an angle of about 30° to about 60° to generate shear stress on the one or more non-red blood cell blood components.

15. The method of claim 14, wherein agitating the isolation container comprises agitation at an angle of about 45° to generate shear stress on the one or more non-red blood cell blood components.

16. (canceled)

17. A method for isolating exosomes or extracellular vesicles from a platelet-rich plasma (PRP) sample, the method comprising:
(a) adding a PRP sample to an isolation container comprising:
a plurality of particles configured to generate shear stress on non-red blood cell blood components; and
an anticoagulant; and
(b) agitating the isolation container to generate shear stress on one or more non-red blood cell blood components and release exosomes or extracellular vesicles from the PRP sample, thereby generating an exosome-rich PRP (ER-PRP).

18. The method of claim 17, further comprising filtering the ER-PRP to remove platelets and generate an exosome-rich plasma (ERP).

19. The method of claim 18, wherein filtering comprises the use of a filter having pores ranging from about 0.1 μm to about 5 μm in size.

20-22. (canceled)

23. A system for isolating exosomes or extracellular vesicles from a blood sample, the system comprising:
an isolation container comprising a physical barrier configured to separate non-red blood cell blood components from red blood cells in a blood sample;
a plurality of particles having a particle density that is less than a density of the physical barrier, the plurality of particles being configured to generate shear stress on non-red blood cell blood components; and
an anticoagulant.

24. (canceled)

25. The system of claim 23, further comprising an agitator to generate shear stress on non-red blood cell blood components.

26-28. (canceled)
